SEQUENCE LISTING (6 sequences)

SEQ ID NO: 1 - DNA, 1473 bp, Arabidopsis thaliana
atggacacttcttcttcacttttgttcttctccttcttcttctttatcatcatcgtcatcttcaacaagatcaacggtctcagatcatccccagcttcaaagaaaaaacttaatgatcatcatgttacatcccagagtcacggaccaaagtttccacacggaagcttgggatggcccgtcatcggtgaaaccatcgagttcgtctcttctgcttactcagaccgtcctgagagtttcatggacaagcgtcgtctcatgtatgggagagtgtttaagtcgcatatttttggaacggcgacgatcgtgtcgacggatgctgaagtgaacagagccgttttacagagcgactcgacagctttcgtgccgttttacccaaaaacggtaagggagctaatgggaaaatcgtcgatacttcttatcaacgggagtttacatagacggttccatggattagtcggttctttcttaaagtcgccacttctcaaagctcaaatcgttagagacatgcacaagtttttgtcggaatccatggatctatggtccgaggaccaacctgtgctcctccaagacgtctccaagactgttgcattcaaagtacttgccaaggcattgataagtgtagagaaaggagaagatttagaagagctaaagagagagtttgaaaatttcatatcaggactcatgtcattaccaattaacttccctggaacgcaactccatagatctctccaagctaagaagaatatggtgaagcaagttgaaagaatcatagaaggcaaaattaggaaaacaaagaacaaggaggaagatgatgttattgcaaaggatgttgtggatgtgttgcttaaggactcaagtgaacatttaactcacaatttgattgctaacaatatgatcgacatgatgatccctggccacgattctgtccctgtcctcattacccttgccgtcaaattcctctctgattctcctgctgccctcaatctcctaacgaaaaacatgaagctgaaaagtttgaaggaattgacaggagagccactatattggaatgactacttgtcgttacctttaacacaaaaggtgattacagagacactgagaatgggaaatgttataattggagtgatgagaaaggcgatgaaagatgttgaaataaaaggatatgtgataccaaaaggatggtgtttcttggcctatctcagatcagttcatcttgatgaagcttattatgagtctccgtacaaatttaatccctggagatggcaagaaagggacatgaacacgagtagtttcagtccttttggaggtggtcagagattgtgccctggtctcgatttggctcgtcttgaaacttcagtttttcttcaccatcttgtcactcgcttcagatggatagcagaagaagacacaatcataaacttcccaacggtgcatatgaagaacaaattacccatttggatcaaaagaatataa

SEQ ID NO: 2 - protein, 490 aa, Arabidopsis thaliana
MDTSSSLLFFSFFFFIIIVIFNKINGLRSSPASKKKLNDHHVTSQSHGPKFPHGSLGWPVIGETIEFVSSAYSDRPESFMDKRRLMYGRVFKSHIFGTATIVSTDAEVNRAVLQSDSTAFVPFYPKTVRELMGKSSILLINGSLHRRFHGLVGSFLKSPLLKAQIVRDMHKFLSESMDLWSEDQPVLLQDVSKTVAFKVLAKALISVEKGEDLEELKREFENFISGLMSLPINFPGTQLHRSLQAKKNMVKQVERIIEGKIRKTKNKEEDDVIAKDVVDVLLKDSSEHLTHNLIANNMIDMMIPGHDSVPVLITLAVKFLSDSPAALNLLTKNMKLKSLKELTGEPLYWNDYLSLPLTQKVITETLRMGNVIIGVMRKAMKDVEIKGYVIPKGWCFLAYLRSVHLDEAYYESPYKFNPWRWQERDMNTSSFSPFGGGQRLCPGLDLARLETSVFLHHLVTRFRWIAEEDTIINFPTVHMKNKLPIWIKRI

SEQ ID NO: 3 - DNA, 1934 bp, Arabidopsis thaliana
  misc_feature (1748)..(1748): n means A, C, G or T.
tgtgcttaggcatatagttattcccaagaaaccggtttaactgtttacgtatgcaacctccggcaagcgcaggacttttccggtcgccggaaaatctcccttggccttataattacatggattatttggtcgctggtttcttggttttgacggccggaatacttctccgtccatggctctggtttcgtctacgaaactcgaaaacgaaagatggagatgaagaagaagataatgaggagaagaagaagggaatgattccaaacggaagcttaggctggccggtgatcggagaaaccctaaacttcatcgcttgtggttattcttctcggcctgttaccttcatggacaaacgaaagtctttatacgggaaagtgttcaaaacgaacataatagggacaccaatcataatatcaaccgatgcagaggtgaataaagtggtgctccaaaaccatgggaacacatttgtccctgcataccctaaatcaattacggaactacttggagaaaactctattctcagcatcaatggacctcatcaaaaaaggcttcacacgctcattggcgcgttcctcagatctcctcacctcaaagaccggatcactcgagacattgaggcctcggttgttctcactttggcgtcttgggctcaacttccattggttcatgttcaggatgagatcaaaaagatgacgtttgagatattagtaaaagtgttgatgagcacatctcctggtgaagatatgaacattctcaaacttgagttcgaagaattcatcaaaggtttgatttgtatcccaatcaaattccctggcactagactctacaaatccttaaaggcgaaagagaggttaataaagatggtaaaaaaggttgtggaggagagacaagtggcgatgacaacgacgtctccggcaaatgacgtggtggacgtacttctaagagacggtggtgattcagagaagcaatctcaaccgtcagatttcgtcagcggaaagatcgtagagatgatgatacccggagaggaaacaatgccaacggcgatgaccttggctgtcaaattcttaagtgacaaccccgtcgctctagccaaactcgtggaggagaatatggagatgaagaggcgtaaattggaattgggagaagaatacaagtggaccgattatatgtctctctcttttactcaaaatgtgataaacgaaacgcttagaatggctaacattattaacggggtgtggaggaaagctctcaaggatgtagaaattaaaggttacttaataccgaaaggatggtgtgtattggcatcattcatatcggttcacatggatgaagacatttatgataatccctatcaattcgatccgtggagatgggacagaattaatggatcggcaaacagcagtatttgcttcacaccctttggtggtgggcaaaggctatgtcctggtttagagctgtcgaagctcgaaatatccatctttcttcaccaccttgtaacccggtacagttggacggctgaggaagacgagatagtgtcatttccgactgtgaagatgaagcggaggctcccgatccgagtggctactgtagatgatagtgcttctccgatctcacttgaagatcattaatagatcatttcaaagaacaaaactgtttgtgcaaagaggaagcagagaagtaaacaaatgatcttattaacaaatagtagagaagagaagcaaacaagattggtgggtaagacagaaagaacnaaacgtacagctagtgatggctcaaagatgagagattctaattataattttttttgtttgtcatgtcaaattataagcgttggttaggttgtccctttctcttttatttatcgtaccaaacgcaagttgagatatgattccatatatatggatgatagatatgtatattaatatatagcggccggg

SEQ ID NO: 4 - protein, 524 aa, Arabidopsis thaliana
MQPPASAGLFRSPENLPWPYNYMDYLVAGFLVLTAGILLRPWLWFRLRNSKTKDGDEEEDNEEKKKGMIPNGSLGWPVIGETLNFIACGYSSRPVTFMDKRKSLYGKVFKTNIIGTPIIISTDAEVNKVVLQNHGNTFVPAYPKSITELLGENSILSINGPHQKRLHTLIGAFLRSPHLKDRITRDIEASVVLTLASWAQLPLVHVQDEIKKMTFEILVKVLMSTSPGEDMNILKLEFEEFIKGLICIPIKFPGTRLYKSLKAKERLIKMVKKVVEERQVAMTTTSPANDVVDVLLRDGGDSEKQSQPSDFVSGKIVEMMIPGEETMPTAMTLAVKFLSDNPVALAKLVEENMEMKRRKLELGEEYKWTDYMSLSFTQNVINETLRMANIINGVWRKALKDVEIKGYLIPKGWCVLASFISVHMDEDIYDNPYQFDPWRWDRINGSANSSICFTPFGGGQRLCPGLELSKLEISIFLHHLVTRYSWTAEEDEIVSFPTVKMKRRLPIRVATVDDSASPISLEDH

SEQ ID NO: 5 - DNA, 20 bp, Artificial sequence
  PCR amplification primer
gttaaaacactaatggacac

SEQ ID NO: 6 - DNA, 21 bp, Artificial sequence
  PCR amplification primer
tgatttatattcttttgatcc

(19) United States
(12) Patent Application Publication
(10) Pub. No.: US 2005/0246797 A1
(43) Pub. Date: Nov. 3, 2005

(54) Gene participating in the synthesis of brassinosteroid

(76) Inventors: Yuichi Tsukaya, Okazaki-shi (JP); Gyung-Tae Kim, Saha-gu (KR)

Correspondence Address: NATH & ASSOCIATES, 1030 15th STREET, NW, 6TH FLOOR, WASHINGTON, DC, 20005, US

(21) Appl. No.: 10/507,106

(22) PCT Filed: Mar. 7, 2003
(86) PCT No.: PCT/JP03/02755

§ 371 Date: Sep. 10, 2004

(30) Foreign Application Priority Data
Mar. 12, 2002 (JP) 2002-67063
Aug. 28, 2002 (JP) 2002-248910

(51) Int. Cl. C12N 15/82; C07H 21/04; C07K 2/00

(52) U.S. Cl. 800/290; 536/23.6; 435/320.1; 530/370; 800/298

(57) ABSTRACT

Brassinosteriods are a kind of plant hormones, which are ubiquitously distributed throughout the plant kingdom and are functional in cell elongation and cell division at extremely low concentrations. However, the most important synthetic enzyme proteins and nucleic acids encoding the proteins regulating the final step of brassinosteroid biosynthesis have not been known. The inventors searched homological nucleotide sequences to ROT3, which the inventors had previously discovered, and found a nucleotide sequence that exhibits 51% identity to ROT3 gene. Examining the sequence, the inventors discovered that the sequence is a novel gene (CYP90D1, SEQ ID NO: 1), which encodes a factor regulating the final step of brassinosteroid biosynthesis, physiologically functional in regulating the size of plant. Furthermore, the inventors discovered that the CYP90D1 gene regulates the final step of the brassinosteroid biosynthesis in combination with ROT3 (=CYP90C1) gene, then accomplished the present invention.

### FIELD OF THE INVENTION

Present invention relates to a gene participating in the synthesis of brassinosteroid, more specifically, to a novel gene (CYP90D1, SEQ ID NO: 1) controlling the final step of synthesis of brassinosteroid in combination with ROT3 gene (=CYP90C1, #51 to #1625 of SEQ ID NO: 3).

### PRIOR ART

Brassinosteriods are a kind of plant hormones, which are ubiquitously distributed throughout the plant kingdom and are functional in cell elongation and cell division at extremely low concentrations, and are generic name of more than 40 kinds of analogues.

Because of their strong action on plants, brassinosteroids have been suggested to be important in their applicability to agricultural industry and many patents related to them have been disclosed (e.g., Japanese Unexamined Patent Publications, 5-222090, 6-98648, 6-340689, 8-59408, 8-81310, 8-113503, 9-97).

Researches on brassinosteroid biosynthesis have been made aggressively and the progressed elucidation of biosynthetic pathways (e.g., Fujioka et al., “Brassinosteroid biosynthesis and signal transduction” in Signal Transduction of Plant Hormones p180-189, Cell Technology Supplement, Plant Cell Technology Series 10, Shujunsha Co. Ltd, (1998)) suggests that cytochrome P450-type proteins regulate the brassinosteroid biosynthesis in plant.

The inventors already identified ROTUNDIFOLIA3 (ROT3) gene, which belongs to a family of cytochromes P450, in Arabidopsis (Gene & Development 12:2381-2391 (1998)), and showed that modulation of the expression of ROT3 gene resulted in morphological alterations of leaves and flowers (Proc. Natl. Acad. Sci. USA vol. 96, pp. 9433-9437 (1999)).

### PROBLEMS TO BE SOLVED BY THE INVENTION

As described above, nucleic acid molecules encoding cytochrome P450-type proteins, which are involved in brassinosteroid biosynthesis, have been identified (published Japanese translation of PCT international publication for patent application (WO97/35986) No. 2000-508524). However, nucleic acid molecules previously known for the biosynthetic pathway are involved in regulation of the steps in comparatively early stage in brassinosteroid biosynthesis. Therefore, it was difficult to apply the action of the above-described nucleic acid molecules to the organ specific control or to the quantitative regulation. Furthermore, neither the enzyme proteins nor nucleic acids encoding the proteins, regulating the final step of brassinosteroid biosynthesis, have been known. The final step as used herein means the step to synthesize brasssinolide (the formula described below) from castasterone (The whole synthetic pathway of brassinosteroid is shown in FIG. 1).

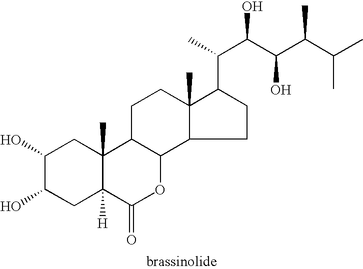

### MEANS TO SOLVE THE PROBLEMS AND DETAILED DESCRIPTION OF THE INVENTION

The inventors searched homological nucleotide sequences to ROT3, which the inventors had previously discovered, and found a nucleotide sequence that exhibits 51% identity to ROT3 gene. Examining the sequence, the inventors discovered that the sequence is a novel gene (CYP90D1, SEQ ID NO: 1), which encodes a factor regulating the final step of the brassinosteroid biosynthesis, physiologically functional in regulating the size of plant. Furthermore, the inventors discovered that the CYP90D 1 gene regulates the final step of the brassinosteroid biosynthesis in combination with ROT3 (=CYP90C1) gene, then accomplished the present invention.

The present invention makes it possible to regulate the biosynthetic pathway of physiologically active brassinosteroid using ROT3 (=CYP90C1) and CYP90D1 and this possibility of regulation is the unique point that differentiates the invention from previous findings.

In other words, the expression of sole ROT3 (=CYP90C1) in a whole plant is effective only to leaves and floral organs, particularly effective in longitudinal direction. Floral organ are derived from deformed leaves, there are common pathways between them in morphological regulation by genes. On the other hand, the combination of ROT3 (=CYP90C1) and CYP90D1 is effective to a whole plant. By manipulating nucleic acid molecules of ROT3 (=CYP90Cl) and CYP90D1 and proteins coded by these genes, the shape of leaves and flowers can be changed at will and at the same time, and also only the shape of flowers can be changed without changing the major part of the height of plants and the shape of leaves.

That is to say, the present invention is a gene (A) having the nucleotide sequence of (1) or (2):
(1) Nucleotide sequence of SEQ ID NO: 1.
(2) Nucleotide sequences encoding either of the following proteins,
   (a) A protein having the amino acid sequence of SEQ ID NO: 2.
   (b) A protein having the amino acid sequence derived from SEQ ID NO: 2, wherein one or some amino acids are deleted, substituted or added and its expression stimulates brassinosteroid biosynthesis.

Furthermore, the present invention is also a polynucleotide (B) having the nucleotide sequence of (1) or (2), and that of (3) or (4):
(1) Nucleotide sequence of SEQ ID NO: 1.
(2) Nucleotide sequences encoding either of the following proteins,
   (a) A protein having the amino acid sequence of SEQ ID NO: 2.
   (b) A protein having the amino acid of SEQ ID No: 2, wherein one or some amino acids are deleted, substituted or added and its expression stimulates brassinosteroid biosynthesis.
(3) Nucleotide sequence of #51 to #1625 of SEQ ID NO: 3.
(4) Nucleotide sequence encoding either of the following proteins,
   (c) A protein having the amino acid sequence of SEQ ID NO: 4.
   (d) A protein having the amino acid sequence of SEQ ID NO: 4, wherein one or some amino acids are deleted, substituted or added and its expression stimulates brassinosteroid biosynthesis.

Moreover, the present invention is i) a polynucleotide comprising a promoter and the gene (A), whose nucleotide sequence is linked to said promoter in forward direction, ii) a polynucleotide comprising a promoter and the gene or a part of the gene (A), whose nucleotide sequence or a part of the sequence is linked to said promoter in reverse direction, iii) a polynucleotide comprising a promoter and the polynucleotide (B), wherein both of the above-described nucleotide sequences are linked to said promoter in forward direction, or iv) a polynucleotide comprising a promoter and the polynucleotide (B) or a part of them, wherein at least one of nucleotide sequence of the above-described nucleotides or a part of them is linked to the above-described promoter in reverse direction.

The promoter used herein will be described later in detail and includes the cauliflower mosaic virus 35S promoter, heat shock promoter, chemical-inducible promoters and others. Additionally, there are no limits on the way to link a promoter with the above-described gene and the linking can be operated appropriately using conventional techniques of genetic engineering.

Still furthermore, the present invention is a plasmid containing either of the above-described genes or the above-described polynucleotides and is also a plant transformed by either of the above-described genes or the above-described polynucleotides.

Still moreover, the present invention is a plasmid containing the above-described polynucleotide. The plasmids used herein include such binary vectors as pBI-121 plasmid, Ti plasmid and others.

Also, the plants applicable by the present invention cover whole Spermatophyte.

To transform such plants, the gene of the present invention was inserted to the above-described plasmid, which may transform the above-described plants using conventional genetic engineering methods.

In addition, the present invention is a method for altering the morphology of a plant, comprising the steps of transforming a plant by the gene (A) or by the polynucleotide (B) and enhancing or suppressing the expression of the above-described gene or the above-described polynucleotide. Furthermore, the present invention is a method for altering the morphology of a plant, which is transformed by any of the above-described genes or polunucleotides, comprising the step of stimulating the responsible promoter in the transformed plant. And also, the present invention is the plant with a morphology altered by any of the above-described methods.

Also, the present invention is a protein of the following (a) or (b):
(a) A protein having the amino acid of SEQ ID NO: 2.
(b) A protein having the amino acid sequence of SEQ ID NO: 2, wherein one or some amino acids are deleted, substituted or added and its expression stimulates brassinosteroid biosynthesis.

Furthermore, the present invention is a mixture or a complex of the above-described protein and a protein of the following (c) or (d):
(c) A protein having the amino acid of SEQ ID NO: 4.
(d) A protein having the amino acid sequence of SEQ ID NO: 4, wherein one or some amino acids are deleted, substituted or added and its expression stimulates brassinosteroid biosynthesis.

It is possible to manipulate nucleic acid molecules of CYP90D 1 and ROT3 (=CYP90C1) and proteins coded by these using the following procedures:
(1) The procedure comprises the steps of linking a manipulable promoter with DNA molecules of CYP90D1 (SEQ ID NO: 1) and ROT3 (=CYP90C1, #51 to #1625 of SEQ ID NO: 3), transducing these into a plant by way of an appropriate conventional method such as Ti plasmid, and giving external stimulation to the promoter to regulate the expression of the above-described genes. The examples of the promoters usable herein are as follows:
   A 35S promoter (possible to express constitutively)
   A heat shock promoter (possible to express temperature dependently)
   A Dex-inducible promoter (possible to express by the exposure to Dexamethason)]
   A CHS-A promoter of petunia (petal specific expression in plants with colored petals and sugar-dependent expression not specific to petal but to leaf bud in Arabitopsis).
      In addition to these promoters, other conventional promoters in plant field are usable.
(2) A method to suppress the function of ROT3 or CYP90D 1:

It is possible to suppress functions of a specific gene by an antisence RNA method (a method for transducing an altered gene so as to be transcribed in reverse direction) or by a RNAi method (a method for transducing an altered gene so as to be ligated a part of a gene tamdemly in forward and reverse and as to be transcribed throughout). The present invention applied the above-described method for suppressing genetic expression. Since the target genes are CYP90D1 (SEQ ID: 1) and ROT3 (=CYP90C1, #51 to #1625 of SEQ ID NO: 3) are revealed, targeted suppression of their expression is possible.
(3) A method of Combination:

It is prerequisite to prepare singly altered strains of CYP90D 1 and ROT3 (=CYP90C1, #51 to #1625 of SEQ ID NO: 3), independently, then, two methods are possible: either crossing between them by classical genetics or direct transduction of both altered genes should bring about doubly altered strains.
(4) A method of precursor fermentation:

There are successful examples for at least a part of genes responsible to brassinosteroid biosynthesis showing enzymatic activity when these genes are expressed in yeast cells. Using the methods of these examples and providing appropriate precursors, it is possible to artificially synthesize castasterone or brassinolide (the final and active product) in yeast cells or in eucaryotic cells, wherein combination of ROT3 and CYO90D1 or one of the above-described genes are expressed.

### BRIEF DESCRIPTION OF DRAWINGS

FIG. 1 shows the whole pathway of brassinosteroid biosynthesis.

FIG. 2 shows morphology of leaves of wild type (Ws-2)(No. 1), a strain of reference example 1 (suppression of ROT3 function) (No.2), a strain of reference example 2 (suppression of both ROT3 and CYP90D1 function)(No. 3 and 4) of Arabidopsis cultivated in the same condition. No.3 and No.4 show a partly effective and a strongly effective strain, respectively.

FIG. 3 shows morphology of leaves of strains without function of ROT3 and CYP90D1 after the treatment with intermediates of brassinosteroid synthesis and brassinolide. Control: no treatment, 6-D-CT: treated with (hereinafter the same) 6-Deoxocathasterone, 6-D-TE:6-Deoxoteasterone, 6-D-3DT: 3-Dehydro-6-deoxoteasterone, 6-D-TY: 6-Deoxotyphasterol 6-D-CS: 6-Deoxocastasterone, CT: Cathasterone, TE: Teasterone 3DT: 3-Dehydroteasterone, TY: Typhasterol, CS: Castasterone, BL :Brassinolide.

### EFFECTS OF THE INVENTION

There are several practical defects in previous invention on the regulation of steps in biosynthetic pathway of steroid compounds, which show distinctive physiological activity in plants.

Namely, the regulatory factor of brassinosteroid biosynthesis previously elucidated is involved in the early steps of the biosynthetic pathway and enforced expression of the factor in transgenic plants brings about spindly growth of the whole plant and enlarges the plant. Therefore, there are no practical utility values for the above-described regulatory factor except for their occasional application. On the other hand, stopping a biosynthetic pathway in a transgenic plant resulted in miniaturization of the whole plant and, again, there are no practical utility except for their occasional application. In other words, conventional methods change the whole shape of a plant, which is practically not valuable. Practically usable and valuable transgenic plants are shown by the following examples: in horticulture, only the size of floral organs is large or only the size of leaves is small, or in improvement of vegetables, only the size of leaves is large. Therefore, conventional methods have difficulty in applying to biodesign of plants without the combination with a special expression-regulatory system. According to the present invention, in contrast to them, it becomes possible to control the size of a specific organ in a specific direction (specially in longitudinal direction) and the whole size of a plant by using the combination of ROT3 (=CYP90C1) with CYP90D1.

Furthermore, the present invention elucidated that ROT3 (=CYP90C1) and CYP90D1 cooperatively regulate the final step of brassinosteroid biosynthesis. Therefore, the invention could be used for various industrial applications using as chemical synthesis of brassinosteriod.

The following examples illustrate this invention, however, it is not intended to limit the scope of the invention.

### MANUFACTURING EXAMPLE 1

As the strain knocked down the function of ROT3, the inventors used rot3- 1 null mutant of Arabidopsis (Tsuge et al., Development 122: 1589-1600 (1996), a functional defect mutant of ROT3. The mutant cell line was seeded under sterilized conditions and cultured at 23° C. under continuous illumination.

### MANUFACTURING EXAMPLE 2

To get the strain knocked down the function of both ROT3 and CYP90D1, the inventors, first of all, isolated cDNA of CYP90D1 from Arabidopsis using a primer set, ROT3h-cDNA-for: 5′-GTTAAAACACTAATGGACAC-3′(SEQ ID NO: 5); ROT3h-cDNA-rev: 5′-TGATTTATATTCTTTTGATCC-3′(SEQ ID NO: 6), which could specifically amplify the ROT3 homologue (CYP90D1). Then, the above-described CYP90D1 (SEQ ID NO: 1) clone was inserted to be transcribed in reverse direction from cauliflower mosaic virus 35S promoter into multipurpose vector pBI121, wherein hygromycin resistant gene was inserted as a selection marker and GUS protein coding region was removed. The construct was transduced into Agrobacterium (C58C1 Rif-resistant) and was introduced into Arabidopsis rot3-1 by in planta method, using conventional way of suspension culture medium of Agrobacterium. After the transformants were selected by hygromycin, transformants with homozygous inserted genes were isolated by self-pollination. Then, the strain was seeded under sterilized conditions and was cultured at 23° C. under continuous illumination.

### EXAMPLE 1

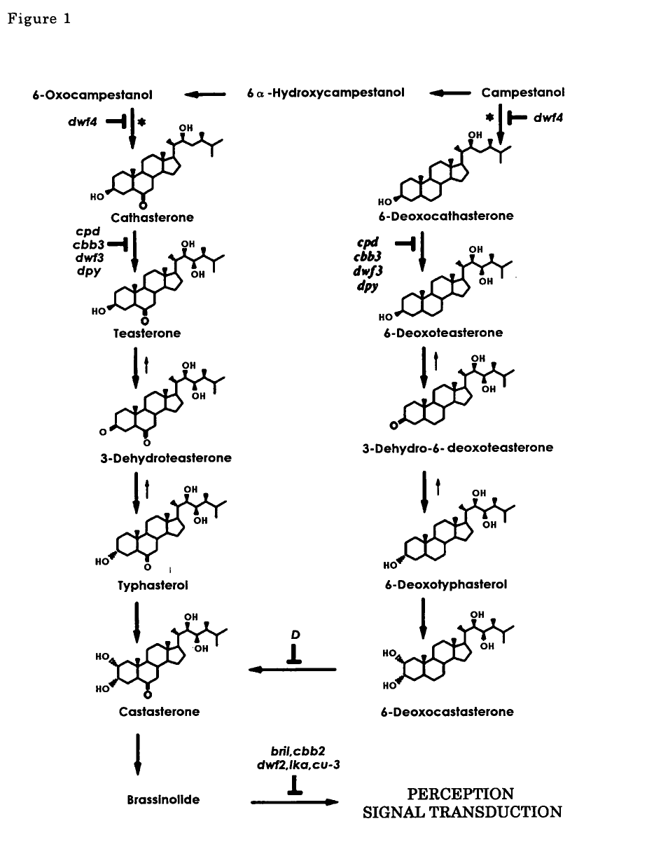

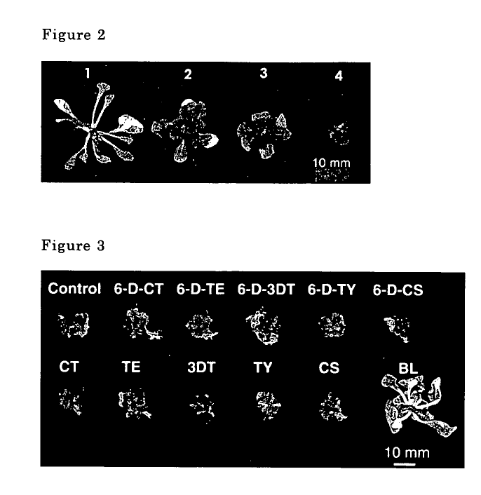

FIG. 2 shows morphology of leaves of wild species (Ws-2)(No. 1), a strain of reference example 1 (suppression of R03 function) (No.2), a strain of reference example 2 (suppression of both Rot3 and CYP90D1 function)(No. 3 and 4) of Arabidopsis cultivated in the same condition. While the leaves of the strain with suppressed function of ROT3 (FIG. 2-2) are shorter in longitudinal direction compared to those of wild type (FIG. 2-1), the leaves of the strain with suppressed function of both ROT3 and CYP90D1 (FIGS. 2-3 & 4) are shorter further more than those of the above strains. In short, ROT3 and CYP90D1 are genes, which are cooperatively involved in biosynthetic pathway of brassinosteroid.

### EXAMPLE 2

The strain with suppressed function of both ROT3 and CYP90D1 prepared in reference example 2 was cultured from seeds in sterilized conditions. The seeds of the above-described strain were seeded on the MS medium (solidified by 0.2% Gelrite) supplemented with 2% (w/v) sucrose and were conventionally cultured at 23° C. under continuous illumination after seeding under sterilized conditions.

On the other hand, aqueous solution (0.1 μM) of intermediates of brassinosteroid biosynthesis (e.g., 6-D-CT: 6-Deoxocathasterone,6-D-TE: 6-Deoxoteasterone,6-D-3DT: 3-Dehydro-6-deoxoteasterone, 6-D-TY: 6-Deoxotyphasterol, 6-D-CS: 6-Deoxocastasterone, CT: Cathasterone, TE:Teasterone 3DT: 3-Dehydroteasterone, TY:Typhasterol, CS:Castasterone) and brassinolide (BL) (BL was obtained from WAKO pharmaceutical Co. (made by FujiKagaku Industry, Co.) and other brassinosteroids were gifts from Dr. Shouzou Jujioka, RIKEN and Dr. Tohide Takatsu, Jouetsu University of Education) was prepared.

The above-described plants (strains of suppressed function of both ROT3 and CYP90D1) were cultured in the above-described aqueous solution at the level of submersion under the solution with gently shaking. In the case of the treatment of leaves with the above-described intermediates or brassionolide, leafstalks were cut out by a surgical knife after taking out the plants under sterilized conditions and were treated in the same way. FIG. 3 shows the photographs of these leaves. As shown in FIG. 3, each brassinosteroid intermediate was not effective to plants without the function of ROT3 and CYP90D1, however, brassinolide (BL), the final product, induced large size of leaves and showed distinguished effects. Namely, ROT3 and CYP90D1 are cooperatively involved in the synthesis of brassinolide, the final product of the biosynthetic pathway of brassinosteriods.

### EXAMPLE 3

The concentrations of brassinosteroids were determined in wild strains (Ws-2), the strain of reference example 1 (rot3-1 and rot3-5, suppressed function of ROT3) and the strain of reference example 3 (rot3/CYP90D1, suppressed function of ROT3 and CYP90D1) of Arabidoposis. The concentration was determined by harvesting the ground part of the plants at the time of rosette formation by reaping, by freezing and drying, and by detecting using HPLC and GC-MS. The results are shown in Table 1.

TABLE 1

| | ng/g | | | |
|---|---|---|---|---|
| | Ws-2 | rot3-1 | rot3-5 | rot3/CYP90D1 |
| 6-Deoxoteasterone | 0.05 | 0.19 | 0.11 | 0.26 |
| 3-Deoxotyphasterol | 2.30 | 3.49 | 4.30 | 0.38 |
| 6-Deoxocastasterone | 2.60 | 1.88 | 4.00 | 0.034 |
| Teasterone | — | 0.004 | 0.02 | — |
| Typhasterol | 0.27 | 0.38 | 0.46 | 0.014 |
| Castasterone | 0.28 | 0.31 | 0.50 | 0.020 |
| Brassinolide | 0.20 | 0.04 | 0.06 | — |

Note:
— in the table shows that the value is less than the limit of detection (0.001 ng/g).

As shown in the table, in the strain (rot3-1 and rot3-5) with suppressed ROT3, the production of brassinolide decreased remarkably, as a consequence of this, the production of brassinosteroids in the previous stage (especially castasterone) increased. However, suppression of ROT3 did not completely block the production of brassinolide. In other words, ROT3 itself dose not completely regulate the biosynthesis of brassinolide.

On the other hand, in the strain (rot3/CYP90D1) with suppressed function of both ROT3 and CYP90D1, the amount of brassinolide extremely decreased among intermediates of brassionosteroid biosynthesis, which indicates that the pathway of the synthesis of brassinolide from castasterone is completely blocked by simultaneous suppression of both ROT3 and CYP90D1. In other words, the production of brassinolide is completely regulated by the combined action of ROT3 and CYP90D1.

1. (canceled)

2. A polynucleotide having the nucleotide sequence of (i) or (2), and that of (3) or (4):
(1) Nucleotide sequence of SEQ ID NO: 1.
(2) Nucleotide sequences encoding either of the following proteins,
(a) A protein having the amino acid sequence of SEQ ID NO: 2.
(b) A protein having the amino acid of SEQ ID No: 2, wherein one or some amino acids are deleted, substituted or added and its expression stimulates brassinosteroid biosynthesis.
(3) Nucleotide sequence of #51 to # 1625 of SEQ ID NO: 3.
(4) Nucleotide sequence encoding either of the following proteins,
(c) A protein having the amino acid sequence of SEQ ID NO: 4.
(d) A protein having the amino acid sequence of SEQ ID NO: 4, wherein one or some amino acids are deleted, substituted or added and its expression stimulates brassinosteroid biosynthesis.

3. (canceled)

4. (canceled)

5. A polynucleotide comprising a promoter and the polynucleotide of claim 2, wherein both of said nucleotide sequences are linked to said promoter in forward direction.

6. A polynucleotide comprising a promoter and the polynucleotide or a part of the polynucleotides of claim 2, wherein at least one of said nucleotide sequence or a part of them is linked to said promoter in reverse direction.

7. A plasmid comprising a gene or the polynucleotide according to any one of claims 2, 5 and 6.

8. A plant transformed by the gene or the polynucleotide according to any one of claims 2, 5 and 6.

9. A method for changing the morphology of a plant, comprising the steps of transforming a plant by the polynucleotide of claim 2, and promoting or suppressing the expression of said gene or said polynucleotide.

10. A method for changing the morphology of a plant, comprising stimulating the promoter of the plant, which is transformed by the gene or the polynucleotide according to claim 5 or 6.

11. The plant with a morphology altered by the method of claim 9.

12. (canceled)

13. A mixture or a complex of a protein of the following (a) or (b) and a protein of the following (c) or (d):
(a) A protein having the amino acid of SEQ ID NO: 2.
(b) A protein having the amino acid sequence of SEQ ID NO: 2, wherein one or some amino acids are deleted, substituted or added and its expression stimulates brassinosteroid biosynthesis.
(c) A protein having the amino acid of SEQ ID NO: 4.
(d) A protein having the amino acid sequence of SEQ ID NO: 4, wherein one or some amino acids are deleted, substituted or added and its expression stimulates the biosynthesis of brassinosteroid.

14. The plant with a morphology altered by the method of claim 10.
